(19) United States
(12) Patent Application Publication
(10) Pub. No.: US 2007/0021498 A1
(43) Pub. Date: Jan. 25, 2007

(54) Novel tricyclic spiroderivatives as modulators of chemokine receptor activity

(76) Inventor: Nafizal Hossain, Lund (SE)

Correspondence Address: FISH & RICHARDSON P.C., P.O BOX 1022, MINNEAPOLIS, MN, 55440-1022, US

(21) Appl. No.: 10/575,522

(22) PCT Filed: Oct. 14, 2004
(86) PCT No.: PCT/SE04/01476

§ 371 Date: Apr. 12, 2006

(51) Int. Cl. A61K 31/353; C07D 317/72

(52) U.S. Cl. 514/462; 549/336

(57) ABSTRACT

The invention provides compounds of formula (I) wherein m, R¹, n, R², q, X, Y, Z, R³, R⁴, R⁵, R⁶, R⁷, R⁸, t and R⁹ are as defined in the specification, processes for their preparation, pharmaceutical compositions containing them and their use in therapy.

The present invention relates to novel compounds, processes for their preparation, pharmaceutical compositions containing them and their use in therapy.

Chemokines play an important role in immune and inflammatory responses in various diseases and disorders, including asthma and allergic diseases, as well as autoimmune pathologies such as rheumatoid arthritis and atherosclerosis. These small secreted molecules are a growing superfamily of 8-14 kDa proteins characterised by a conserved four cysteine motif. The chemokine superfamily can be divided into two main groups exhibiting characteristic structural motifs, the Cys-X-Cys (C-X-C) and Cys-Cys (C-C) families. These are distinguished on the basis of a single amino acid insertion between the NH-proximal pair of cysteine residues and sequence similarity.

The C-X-C chemokines include several potent chemoattractants and activators of neutrophils such as interleukin-8 (IL-8) and neutrophil-activating peptide 2 (NAP-2).

The C-C chemokines include potent chemoattractants of monocytes and lymphocytes but not neutrophils such as human monocyte chemotactic proteins 1-3 (MCP-1, MCP-2 and MCP-3), RANTES (Regulated on Activation, Normal T Expressed and Secreted), eotaxin and the macrophage inflammatory proteins 1α and 1β (MIP-1α and MIP-1β).

Studies have demonstrated that the actions of the chemokines are mediated by subfamilies of G protein-coupled receptors, among which are the receptors designated CCR1, CCR2, CCR2A, CCR2B, CCR3, CCR4, CCR5, CCR6, CCR7, CCR8, CCR9, CCR10, CXCR1, CXCR2, CXCR3 and CXCR4. These receptors represent good targets for drug development since agents which modulate these receptors would be useful in the treatment of disorders and diseases such as those mentioned above.

In accordance with the present invention, there is therefore provided a compound of formula 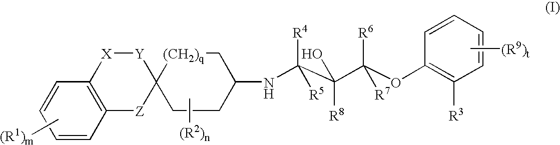
wherein
m is 0, 1, 2, 3 or 4;
each R¹ independently represents halogen, cyano, hydroxyl, C₁-C₆ alkyl, C₁-C₆ haloalkyl, C₁-C₆ alkoxy or sulphonamido (—SO₂NH₂);
either X represents a bond, —CH₂—, —O— or —C(O)— and Y represents a bond, —CH₂—, —O— or —C(O)—, or X and Y together represent a group —CH═C(CH₃)— or —C(CH₃)═CH—, and Z represents a bond, —O—, —NH— or —CH₂—, provided that only one of X, Y and Z can represent a bond at any one time and provided that X and Y do not both simultaneously represent —— or —C(O)—;
n is 0, 1 or 2;
each R² independently represents halogen or C₁-C₆ alkyl;
q is 0 or 1;
R³ represents —NHC(O)R¹⁰, —C(O)NR¹¹R¹² or —COOR^{12a};
R⁴, R⁵, R⁶, R⁷ and R⁸ each independently represent a hydrogen atom or a C₁-C₆ alkyl group;
t is 0, 1 or 2;
each R⁹ independently represents halogen, cyano, hydroxyl, carboxyl, C₁-C₆ alkoxy, C₁-C₆ alkoxycarbonyl, C₁-C₆ haloalkyl, or C₁-C₆ alkyl optionally substituted by at least one substituent selected from carboxyl and C₁-C₆ alkoxycarbonyl;
R¹⁰ represents a group C₁-C₆ alkyl, C₂-C₆ alkenyl, C₃-C₆ cycloalkyl, adamantyl, C₅-C₆ cycloalkenyl, phenyl or a saturated or unsaturated 5- to 10-membered heterocyclic ring system comprising at least one ring heteroatom selected from nitrogen, oxygen and sulphur, each of which may be optionally substituted by one or more substituents independently selected from nitro, hydroxyl, oxo, halogen, carboxyl, C₁-C₆ alkyl, C₁-C₆ alkoxy, C₁-C₆ alkylthio, C₁-C₆ alkylcarbonyl, Cₗ-C₆ alkoxycarbonyl, phenyl and —NHC(O)—R¹³, or
R¹⁰ represents a group —NR¹⁴R¹⁵ or —O—R¹⁶;
R¹¹ and R¹² each independently represent (i) a hydrogen atom, (ii) a 3- to 6-membered saturated or unsaturated ring optionally comprising at least one ring heteroatom selected from nitrogen, oxygen and sulphur and optionally further comprising a bridging group, the ring being optionally substituted with at least one substituent selected from halogen, hydroxyl, C₁-C₆ alkyl, C₁-C₆ hydroxyalkyl and C₁-C₆ haloalkyl, (iii) a C₁-C₆ alkyl group optionally substituted by at least one substituent selected from halogen, amino (—NH₂), hydroxyl, C₁-C₆ haloalkyl, carboxyl, C₁-C₆ alkoxy, C₁-C₆ alkoxycarbonyl, C₁-C₆ alkylcarbonylamino and a 3- to 6-membered saturated or unsaturated ring optionally comprising at least one ring heteroatom selected from nitrogen, oxygen and sulphur and optionally further comprising a bridging group, the ring being optionally substituted with at least one substituent selected from halogen, hydroxyl, oxo (═O), C₁-C₆ alkyl, C₁-C₆ hydroxyalkyl and C₁-C₆ haloalkyl, or
(iv) C₁-C₆ alkylsulphonyl, or
R¹¹ and R¹² together with the nitrogen atom to which they are attached form a 4- to 7-membered saturated heterocyclic ring that optionally further comprises a ring nitrogen, oxygen or sulphur atom and that is optionally fused to a benzene ring to form a 8- to 11-membered ring system, the heterocyclic ring or ring system being optionally substituted with at least one substituent selected from halogen, hydroxyl, amido (—CONH₂), C₁-C₆ alkyl, C₁-C₆ hydroxyalkyl, C₁-C₆ alkoxy, C₁-C₆ alkoxycarbonyl, C₁-C₆ haloalkyl, C₁-C₆ alkylamino, di-C₁-C₆ alkylamino, C₁-C₆ alkylcarbonyl, C₁-C₆ alkylcarbonylamino, C₁-C₆ alkylaminocarbonyl, di-C₁-C₆ alkylaminocarbonyl, phenyl, halophenyl, phenylcarbonyl, phenylcarbonyloxy and hydroxydiphenylmethyl;
   R^{12a} represents a hydrogen atom or a C₁-C₆ alkyl group;
   R¹³ represents a C₁-C₆ alkyl, amino (—NH₂) or phenyl group;
   R¹⁴ and R¹⁵ each independently represent a hydrogen atom, or a group C₁-C₆ alkyl, C₁-C₆ alkylsulphonyl, phenyl or a saturated or unsaturated 5- to 10-membered heterocyclic ring system comprising at least one ring heteroatom selected from nitrogen, oxygen and sulphur, each group being optionally substituted as defined above for R¹⁰, or
   R¹⁴ and R¹⁵ together with the nitrogen atom to which they are attached form a 4- to 7-membered saturated heterocyclic ring that optionally further comprises a ring nitrogen, oxygen or sulphur atom, the heterocyclic ring being optionally substituted by at least one hydroxyl; and
   R¹⁶ represents a hydrogen atom, or a group C₁-C₆ alkyl, phenyl or a saturated or unsaturated 5- to 10-membered heterocyclic ring system comprising at least one ring heteroatom selected from nitrogen, oxygen and sulphur, each group being optionally substituted as defined above for R¹⁰;
      or a pharmaceutically acceptable salt or solvate thereof.

In the context of the present specification, unless otherwise stated, an alkyl or alkenyl substituent group or an alkyl moiety in a substituent group may be linear or branched. The alkyl moieties in a di-alkylamino or di-alkylaminocarbonyl substituent group may be the same or different. A haloalkyl or halophenyl substituent group will comprise at least one halogen atom, e.g. one, two, three or four halogen atoms. A hydroxyalkyl substituent may contain one or more hydroxyl groups but preferably contains one or two hydroxyl groups. In the ring substituted by R², R² may be attached to any suitable ring carbon atom including the carbon atom of (CH₂)q. When R¹¹ and R¹² or R¹⁴ and R¹⁵ represent a 4- to 7-membered saturated heterocycle, it should be understood that the heterocycle will contain no more than two ring heteroatoms: the nitrogen ring atom to which R¹¹ and R¹² or R¹⁴ and R¹⁵ are attached and optionally a nitrogen, oxygen or sulphur ring atom. In the definition of R¹⁰ (or R¹⁴, R¹⁵ or R¹⁶) it should be noted that the saturated or unsaturated 5- to 10-membered heterocyclic ring system may have alicyclic or aromatic properties. Similarly, in the definition of R¹¹ or R¹², a 3- to 6-membered saturated or unsaturated ring optionally comprising at least one ring heteroatom may have alicyclic or aromatic properties. An unsaturated ring system will be partially or fully unsaturated.

In an embodiment of the invention, m is 0 or 1.

Each R¹ independently represents halogen (e.g. chlorine, fluorine, bromine or iodine), cyano, hydroxyl, C₁-C₆, preferably C₁-C₄, alkyl (e.g. methyl, ethyl, n-propyl, isopropyl, n-butyl, isobutyl, tert-butyl, n-pentyl or n-hexyl), C₁-C₆, preferably C₁-C₄, haloalkyl (e.g. trifluoromethyl), C₁-C₆, preferably C₁-C₄, alkoxy (e.g. methoxy, ethoxy, n-propoxy or n-butoxy) or sulphonamido.

In an embodiment of the invention, each R¹ independently represents halogen, C₁-C₆, preferably C₁-C₄, alkyl or C₁-C₆, preferably C₁-C₄, haloalkyl.

In another embodiment, each R¹ independently represents fluorine, chlorine, methyl or trifluoromethyl, particularly chlorine.

Combinations of X and Y of particular interest include any one or more of the following:

| X | Y |
|---|---|
| bond | O |
| O | bond |
| CH₂ | bond |
| bond | CH₂ |
| CH₂ | O |
| O | CH₂ |
| C(O) | O |
| O | C(O) |
| CH₂ | CH₂ |

—CH═C(CH₃)—

In an embodiment of the invention, X and Y have the meanings shown below:

| X | Y |
|---|---|
| bond | O |
| O | bond |
| CH₂ | O |
| O | CH₂ |
| C(O) | O |
| O | C(O) |
| CH₂ | CH₂ |

—CH═C(CH₃)—

In a further embodiment, X and Y have the meanings shown below:

| X | Y |
|---|---|
| bond | O |
| O | bond |
| CH₂ | bond |
| bond | CH₂ |

In an embodiment of the invention, Z represents a bond, —O— or —CH₂—.

Combinations of X, Y and Z of particular interest include any one or more of the following:

| X | Y | Z |
|---|---|---|
| bond | O | CH₂ |
| O | bond | CH₂ |
| CH₂ | bond | O |
| bond | CH₂ | O |
| CH₂ | O | bond |
| C(O) | O | bond |
| O | C(O) | bond |
| CH₂ | CH₂ | bond |
| O | bond | O |
| bond | O | O |
| CH₂ | CH₂ | O |
| O | CH₂ | CH₂ |

| | |
|---|---|
| —CH═C(CH₃)— | bond |

In an embodiment of the invention, X, Y and Z have the meanings shown below:

| X | Y | Z |
|---|---|---|
| bond | O | CH₂ |
| O | bond | CH₂ |
| CH₂ | O | bond |
| O | CH₂ | bond |
| C(O) | O | bond |
| O | C(O) | bond |
| CH₂ | CH₂ | bond |
| bond | O | O |
| O | bond | O |

| | |
|---|---|
| —CH═C(CH₃)— | bond |

In another embodiment of the invention, X, Y and Z have the meanings shown below:

| X | Y | Z |
|---|---|---|
| bond | O | CH₂ |
| O | bond | CH₂ |
| CH₂ | bond | O |
| bond | CH₂ | O |

Each R² independently represents halogen (e.g. chlorine, fluorine, bromine or iodine) or C₁-C₆, preferably C₁-C₄, alkyl (e.g. methyl, ethyl, n-propyl, isopropyl, n-butyl, isobutyl, tert-butyl, n-pentyl or n-hexyl).

In an embodiment of the invention, n is 1 and R² represents halogen, particularly fluorine.

In an embodiment of the invention, R³ represents —NHC(O)R¹⁰.

In another embodiment of the invention, R³ represents —C(O)NR¹¹R¹².

R⁴, R⁵, R⁶, R⁷ and R⁸ each independently represent a hydrogen atom or a C₁-C₆, preferably C₁-C₄, alkyl group (e.g. methyl, ethyl, n-propyl, isopropyl, n-butyl, isobutyl, tert-butyl, n-pentyl or n-hexyl).

In an embodiment of the invention, R⁴, R⁵, R⁶, R⁷ and R⁸ each independently represent a hydrogen atom or a methyl group.

In another embodiment of the invention, R⁴, R⁵, R⁶ and R⁷ each represent a hydrogen atom and R⁸ represents a methyl group.

In an embodiment of the invention, R⁴, R⁵, R⁶, R⁷ and R⁸ each represent a hydrogen atom.

In an embodiment of the invention, t is 0, 1 or 2, particularly 0 or 1.

Each R⁹ independently represents halogen (e.g. chlorine, fluorine, bromine or iodine), cyano, hydroxyl, carboxyl, C₁-C₆, preferably C₁-C₄, alkoxy (e.g. methoxy, ethoxy, n-propoxy or n-butoxy), C₁-C₆, preferably C₁-C₄, alkoxycarbonyl (e.g. methoxycarbonyl, ethoxycarbonyl, n-propoxycarbonyl or n-butoxycarbonyl), C₁-C₆, preferably C₁-C₄, haloalkyl (e.g. trifluoromethyl), or C₁-C₆, preferably C₁-C₄, alkyl (e.g. methyl, ethyl, n-propyl, isopropyl, n-butyl, isobutyl, tert-butyl, n-pentyl or n-hexyl) optionally substituted by at least one substituent (e.g. one, two or three substituents) independently selected from carboxyl and C₁-C₆, preferably C₁-C₄, alkoxycarbonyl (e.g. methoxycarbonyl, ethoxycarbonyl, n-propoxycarbonyl or n-butoxycarbonyl).

In an embodiment of the invention, each R⁹ independently represents halogen, cyano, hydroxyl, carboxyl, C₁-C₆, preferably C₁-C₄, alkoxy, C₁-C₆, preferably C₁-C₄, alkoxycarbonyl, C₁-C₆, preferably C₁-C₄, haloalkyl or C₁-C₆, preferably C₁-C₄, alkyl.

In another embodiment of the invention, each R⁹ independently represents halogen, hydroxyl, carboxyl, methyl, methoxy, methoxycarbonyl or trifluoromethyl.

In a further embodiment, each R⁹ independently represents halogen (particularly fluorine) or hydroxyl.

R⁹ is preferably bound to a carbon atom located in the para position with respect to the carbon atom to which either the oxygen atom or the group R³ is bound, as indicated by the asterisks in the partial structure shown below:

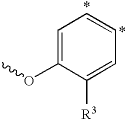

R¹⁰ may represent a group C₁-C₆, preferably C₁-C₄, alkyl group (e.g. methyl, ethyl, n-propyl, isopropyl, n-butyl, isobutyl, tert-butyl, n-pentyl or n-hexyl), C₂-C₆, preferably C₂-C₄, alkenyl, C₃-C₆ cycloalkyl (cyclopropyl, cyclobutyl, cyclopentyl or cyclohexyl), adamantyl, C₅-C₆ cycloalkenyl, phenyl or a saturated or unsaturated 5- to 10-membered heterocyclic ring system comprising at least one ring heteroatom (e.g. one, two, three or four ring heteroatoms independently) selected from nitrogen, oxygen and sulphur, each of which (i.e. each of the recited groups and the ring system) may be optionally substituted by one or more (e.g. one, two, three or four) substituents independently selected from nitro, hydroxyl, oxo, halogen (e.g. fluorine, chlorine, bromine or iodine), carboxyl, C₁-C₆, preferably C₁-C₄, alkyl (e.g. methyl, ethyl, n-propyl, isopropyl, n-butyl, isobutyl, tert-butyl, n-pentyl or n-hexyl), C₁-C₆, preferably C₁-C₄, alkoxy (e.g. methoxy, ethoxy, n-propoxy or n-butoxy), C₁-C₆, preferably C₁-C₄, alkylthio (e.g. methylthio or ethylthio), C₁-C₆, preferably C₁-C₄, alkylcarbonyl (e.g. methylcarbonyl, ethylcarbonyl, n-propylcarbonyl, isopropylcarbonyl, n-butylcarbonyl, n-pentylcarbonyl or n-hexylcarbonyl), C₁-C₆, preferably C₁-C₄, alkoxycarbonyl (e.g. methoxycarbonyl or ethoxycarbonyl), phenyl and —NHC(O)—R¹³.

The saturated or unsaturated 5- to 10-membered heterocyclic ring system in R¹⁰ may be monocyclic or polycyclic (e.g. bicyclic), examples of which include pyrrolidinyl, piperidinyl, pyrazolyl, thiazolidinyl, thienyl, isoxazolyl, thiadiazolyl, pyrrolyl, furanyl, thiazolyl, indolyl, quinolinyl, benzimidazolyl, triazolyl, tetrazolyl, pyridinyl and combinations of any two or more thereof.

In an embodiment of the invention, R¹⁰ represents a group C₁-C₆ alkyl, C₃-C₆ cycloalkyl, phenyl or a saturated or unsaturated 5- to 6-membered heterocyclic ring system comprising at least one ring heteroatom (e.g. one or two ring heteroatoms independently) selected from nitrogen, oxygen and sulphur, each of which (i.e. each of the recited groups and the ring system) may be optionally substituted by one, two, three or four substituents independently selected from nitro, hydroxyl, oxo, halogen, carboxyl, C₁-C₆, preferably C₁-C₄, alkyl, C₁-C₆, preferably C₁-C₄, alkoxy, C₁-C₆, preferably C₁-C₄, alkylthio,

C₁-C₆, preferably C₁-C₄, alkylcarbonyl, C₁-C₆, preferably C₁-C₄, alkoxycarbonyl, phenyl and —NHC(O)—R¹³.

In another embodiment of the invention, R¹⁰ represents a group C₁-C₆ alkyl, C₃-C₆ cycloalkyl or phenyl, each of which may be optionally substituted by one or two substituents independently selected from halogen, C₁-C₆, preferably C₁-C₄, alkyl and C₁-C₆, preferably C₁-C₄, alkoxy.

In still another embodiment of the invention, R¹⁰ represents C₁-C₆ alkyl, cyclopentyl or phenyl, particularly C₁-C₆ alkyl.

Alternatively, R¹⁴ may represent a group —NR¹⁴R¹⁵ or —O—R¹⁶.

R¹⁴ and R¹⁵ each independently represent a hydrogen atom, or a group C₁-C₆, preferably C₁-C₄, alkyl (e.g. methyl, ethyl, n-propyl, isopropyl, n-butyl, isobutyl, tert-butyl, n-pentyl or n-hexyl), C₁-C₆, preferably C₁-C₄, alkylsulphonyl (e.g. methylsulphonyl, ethylsulphonyl, n-propylsulphonyl, isopropylsulphonyl, n-butylsulphonyl, isobutylsulphonyl, tert-butylsulphonyl, n-pentylsulphonyl or n-hexylsulphonyl), phenyl or a saturated or unsaturated 5- to 10-membered heterocyclic ring system comprising at least one ring heteroatom (e.g. one, two, three or four ring heteroatoms independently) selected from nitrogen, oxygen and sulphur, each group (i.e. each of the recited groups including the ring system) being optionally substituted as defined above for R¹⁰ (that is, optionally substituted with one or more (e.g. one, two, three or four) substituents independently selected from nitro, hydroxyl, oxo, halogen (e.g. fluorine, chlorine, bromine or iodine), carboxyl, C₁-C₆, preferably C₁-C₄, alkyl (e.g. methyl, ethyl, n-propyl, isopropyl, n-butyl, isobutyl, tert-butyl, n-pentyl or n-hexyl), C₁-C₆, preferably C₁-C₄, alkoxy (e.g. methoxy, ethoxy, n-propoxy or n-butoxy), C₁-C₆, preferably C₁-C₄, alkylthio (e.g. methylthio or ethylthio), C₁-C₆, preferably C₁-C₄, alkylcarbonyl (e.g. methylcarbonyl, ethylcarbonyl, n-propylcarbonyl, isopropylcarbonyl, n-butylcarbonyl, n-pentylcarbonyl or n-hexylcarbonyl), C₁-C₆, preferably C₁-C₄, alkoxycarbonyl (e.g. methoxycarbonyl or ethoxycarbonyl), phenyl and —NHC(O)—R¹³),
or R¹⁴ and R¹⁵ together with the nitrogen atom to which they are attached form a 4- to 7-membered saturated heterocyclic ring that optionally further comprises a ring nitrogen, oxygen or sulphur atom (e.g. pyrrolidinyl, piperidinyl, morpholino, piperazinyl or thiomorpholinyl), the heterocyclic ring being optionally substituted by at least one hydroxyl (e.g. one or two hydroxyls).

In R¹⁴ or R¹⁵, the saturated or unsaturated 5- to 10-membered heterocyclic ring system may be monocyclic or polycyclic (e.g. bicyclic), examples of which include pyrrolidinyl, piperidinyl, pyrazolyl, thiazolidinyl, thienyl, isoxazolyl, thiadiazolyl, pyrrolyl, furanyl, thiazolyl, indolyl, quinolinyl, benzimidazolyl, triazolyl, tetrazolyl, pyridinyl and combinations of any two or more thereof.

In an embodiment of the invention, R¹⁴ and R¹⁵ each independently represent a hydrogen atom or a C₁-C₆ alkyl or C₁-C₆ alkylsulphonyl group, each group being optionally substituted as defined above for R¹⁰, or R¹⁴ and R¹⁵ together with the nitrogen atom to which they are attached form a 5- to 6-membered saturated heterocyclic ring that optionally further comprises a ring nitrogen, oxygen or sulphur atom, the heterocyclic ring being optionally substituted by at least one hydroxyl.

In a further embodiment, R¹⁴ and R¹⁵ each independently represent a hydrogen atom or a Cₗ-C₆ alkylsulphonyl group, or R¹⁴ and R¹⁵ together with the nitrogen atom to which they are attached form a 5- to 6-membered saturated heterocyclic ring that is optionally substituted by at least one hydroxyl.

In a still further embodiment, R¹⁴ and R¹⁵ each independently represent a hydrogen atom or a methylsulphonyl group, or R¹⁴ and R¹⁵ together with the nitrogen atom to which they are attached form a pyrrolidinyl or piperidinyl ring optionally substituted by one hydroxyl group.

R¹⁶ represents a hydrogen atom, or a group C₁-C₆, preferably C₁-C₄, alkyl (e.g. methyl, ethyl, n-propyl, isopropyl, n-butyl, isobutyl, tert-butyl, n-pentyl or n-hexyl), phenyl or a saturated or unsaturated 5- to 10-membered heterocyclic ring system comprising at least one ring heteroatom (e.g. one, two, three or four ring heteroatoms independently) selected from nitrogen, oxygen and sulphur, each group (i.e. each of the recited groups including the ring system) being optionally substituted as defined above for R¹⁰ (that is, optionally substituted with one or more (e.g. one, two, three or four) substituents independently selected from nitro, hydroxyl, oxo, halogen (e.g. fluorine, chlorine, bromine or iodine), carboxyl, C₁-C₆, preferably C₁-C₄, alkyl (e.g. methyl, ethyl, n-propyl, isopropyl, n-butyl, isobutyl, tert-butyl, n-pentyl or n-hexyl), C₁-C₆, preferably C₁-C₄, alkoxy (e.g. methoxy, ethoxy, n-propoxy or n-butoxy), C₁-C₆, preferably C₁-C₄, alkylthio (e.g. methylthio or ethylthio), C₁-C₆, preferably C₁-C₄, alkylcarbonyl (e.g. methylcarbonyl, ethylcarbonyl, n-propylcarbonyl, isopropylcarbonyl, n-butylcarbonyl, n-pentylcarbonyl or n-hexylcarbonyl), C₁-C₆, preferably C₁-C₄, alkoxycarbonyl (e.g. methoxycarbonyl or ethoxycarbonyl), phenyl and —NHC(O)—R¹³).

In R¹⁶, the saturated or unsaturated 5- to 10-membered heterocyclic ring system may be monocyclic or polycyclic (e.g. bicyclic), examples of which include pyrrolidinyl, piperidinyl, pyrazolyl, thiazolidinyl, thienyl, isoxazolyl, thiadiazolyl, pyrrolyl, furanyl, thiazolyl, indolyl, quinolinyl, benzimidazolyl, triazolyl, tetrazolyl, pyridinyl and combinations of any two or more thereof.

R¹¹ and R¹² each independently represent
(i) a hydrogen atom,
(ii) a 3- to 6-membered saturated or unsaturated ring optionally comprising at least one ring heteroatom (e.g. one, two, three or four ring heteroatoms independently) selected from nitrogen, oxygen and sulphur and optionally further comprising a bridging group (examples of which include cyclopropyl, cyclobutyl, cyclopentyl, cyclohexyl, bicyclo[2.2.1]heptyl, phenyl, pyrolyl, imidazolyl, pyridinyl, pyrazinyl, pyridazinyl, tetrazolyl, pyrimidinyl, thienyl, furanyl, tetrahydrofuranyl and combinations of any two or more thereof), the ring being optionally substituted with at least one substituent (e.g. one, two or three substituents independently) selected from halogen (e.g. fluorine, chlorine, bromine or iodine), hydroxyl, C₁-C₆, preferably C₁-C₅, alkyl (e.g. methyl, ethyl, n-propyl, isopropyl, n-butyl, isobutyl, tert-butyl, n-pentyl, 1,1-dimethylpropyl or n-hexyl), C₁-C₆, preferably C₁-C₄, hydroxyalkyl (e.g. —CH₂OH, —CH₂CH₂OH, —CH₂CH₂CH₂OH or —CH(OH)CH₃) and C₁-C₆, preferably C₁-C₄, haloalkyl (e.g. trifluoromethyl),
(iii) a C₁-C₆ alkyl group optionally substituted by at least one substituent (e.g. one, two, three or four substituents independently) selected from halogen (e.g. fluorine, chlorine, bromine or iodine), amino (—NH₂), hydroxyl, C₁-C₆, preferably C₁-C₄, haloalkyl (e.g. trifluoromethyl), carboxyl, C₁-C₆, preferably C₁-C₄, alkoxy (e.g. methoxy, ethoxy, n-propoxy or n-butoxy), C₁-C₆, preferably C₁-C₄, alkoxycarbonyl (e.g. methoxycarbonyl or ethoxycarbonyl), C₁-C₆, preferably C₁-C₄, alkylcarbonylamino (e.g. methylcarbonylamino or ethylcarbonylamino) and a 3- to 6-membered saturated or unsaturated ring optionally comprising at least one ring heteroatom (e.g. one, two, three or four ring heteroatoms independently) selected from nitrogen, oxygen and sulphur and optionally further comprising a bridging group (examples of which include cyclopropyl, cyclobutyl, cyclopentyl, cyclohexyl, bicyclo[2.2.1]heptyl, phenyl, pyrolyl, imidazolyl, pyridinyl, pyrazinyl, pyridazinyl, tetrazolyl, pyrimidinyl, thienyl, furanyl, tetrahydrofuranyl and combinations of any two or more thereof), the ring being optionally substituted with at least one substituent (e.g. one, two or three substituents independently) selected from halogen (e.g. fluorine, chlorine, bromine or iodine), hydroxyl, oxo (═O), C₁-C₆, preferably C₁-C₄, alkyl (e.g. methyl, ethyl, n-propyl, isopropyl, n-butyl, isobutyl, tert-butyl, n-pentyl or n-hexyl), C₁-C₆, preferably C₁-C₄, hydroxyalkyl (e.g. —CH₂OH, —CH₂CH₂OH, —CH₂CH₂CH₂OH or —CH(OH)CH₃) and C₁-C₆, preferably C₁-C₄, haloalkyl (e.g. trifluoromethyl), or
(iv) C₁-C₆, preferably C₁-C₄, alkylsulphonyl (e.g. methylsulphonyl or ethylsulphonyl), or
R¹¹ and R¹² together with the nitrogen atom to which they are attached form a 4- to 7-membered saturated heterocyclic ring that optionally further comprises a ring nitrogen, oxygen or sulphur atom (e.g. pyrrolidinyl, piperidinyl, piperazinyl, morpholinyl or thiomorpholinyl) and that is optionally fused to a benzene ring to form a 8- to 11-membered ring system (e.g. dihydroisoquinolinyl or dihydroisoindolyl), the heterocyclic ring or ring system being optionally substituted with at least one substituent (e.g. one, two,three or four substituents independently) selected from halogen (e.g. fluorine, chlorine, bromine or iodine), hydroxyl, amido (—CONH₂), C₁-C₆, preferably C₁-C₄, alkyl (e.g. methyl, ethyl, n-propyl, isopropyl, n-butyl, isobutyl, tert-butyl, n-pentyl or n-hexyl), C₁-C₆, preferably C₁-C₄, hydroxyalkyl (e.g. —CH₂OH, —CH₂CH₂OH, —CH₂CH₂CH₂OH or —CH(OH)CH₃), C₁-C₆, preferably C₁-C₄, alkoxy (e.g. methoxy, ethoxy, n-propoxy or n-butoxy), C₁-C₆, preferably C₁-C₄, alkoxycarbonyl (e.g. methoxycarbonyl or ethoxycarbonyl), C₁-C₆, preferably C₁-C₄, haloalkyl (e.g. trifluoromethyl), Cₗ-C₆, preferably C₁-C₄, alkylamino (e.g. methylamino or ethylamino), di-C₁-C₆, preferably C₁-C₄, alkylamino (e.g. dimethylamino), C₁-C₆, preferably C₁-C₄, alkylcarbonyl (e.g. methylcarbonyl or ethylcarbonyl), C₁-C₆, preferably C₁-C₄, alkylcarbonylamino (e.g. methylcarbonylamino or ethylcarbonylamino), C₁-C₆, preferably C₁-C₄, alkylaminocarbonyl (e.g. methylaminocarbonyl or ethylaminocarbonyl), di-C₁-C₆, preferably C₁-C₄, alkylaminocarbonyl (e.g. dimethylaminocarbonyl), phenyl, halophenyl (e.g. fluorophenyl or chlorophenyl), phenylcarbonyl, phenylcarbonyloxy and hydroxydiphenylmethyl.

In an embodiment of the invention, R¹¹ and/or R¹² represents a 3- to 6-membered saturated or unsaturated ring optionally comprising at least one ring heteroatom (e.g. one, two, three or four ring heteroatoms independently) selected from nitrogen, oxygen and sulphur and optionally further comprising a bridging group, the ring being optionally substituted with at least one substituent (e.g. one, two or three substituents independently) selected from hydroxyl, C₁-C₆, preferably C₁-C₅, alkyl (e.g. methyl, ethyl, n-propyl, isopropyl, n-butyl, isobutyl, tert-butyl, n-pentyl, 1,1-dimethylpropyl or n-hexyl) and C₁-C₆, preferably C₁-C₄, hydroxyalkyl (e.g. —CH₂OH, —CH₂CH₂OH, —CH₂CH₂CH₂OH or —CH(OH)CH₃).

In a further embodiment of the invention, R¹¹ and/or R¹² represents a 3- to 6-membered saturated or unsaturated ring optionally comprising at least one ring nitrogen atom and optionally further comprising a bridging group (in particular, cyclopropyl, cyclopentyl, cyclohexyl, bicyclo[2.2.1]heptyl, phenyl, pyrrolidinyl and tetrazolyl), the ring being optionally substituted with at least one substituent (e.g. one, two or three substituents independently) selected from hydroxyl, C₁-C₅ alkyl and C₁-C₂ hydroxyalkyl.

In an embodiment of the invention, R¹¹ and/or R¹² represents a C₁-C₆ alkyl group optionally substituted by at least one substituent (e.g. one, two, three or four substituents independently) selected from amino, hydroxyl, C₁-C₆, preferably C₁-C₄, alkoxy (e.g. methoxy, ethoxy, n-propoxy or n-butoxy), C₁-C₆, preferably C₁-C₄, alkoxycarbonyl (e.g. methoxycarbonyl or ethoxycarbonyl), C₁-C₆, preferably C₁-C₄, alkylcarbonylamino (e.g. methylcarbonylamino or ethylcarbonylamino) and a 3- to 6-membered saturated or unsaturated ring optionally comprising at least one ring heteroatom (e.g. one, two, three or four ring heteroatoms independently) selected from nitrogen and oxygen and optionally further comprising a bridging group, the ring being optionally substituted with at least one substituent (e.g. one, two or three substituents independently) selected from halogen (e.g. fluorine, chlorine, bromine or iodine), hydroxyl, oxo, C₁-C₆, preferably C₁-C₄, alkyl (e.g. methyl, ethyl, n-propyl, isopropyl, n-butyl, isobutyl, tert-butyl, n-pentyl or n-hexyl), C₁-C₆, preferably C₁-C₄, hydroxyalkyl (e.g. —CH₂OH, —CH₂CH₂OH, —CH₂CH₂CH₂OH or —CH(OH)CH₃) and C₁-C₆, preferably C₁-C₄, haloalkyl (e.g. trifluoromethyl).

In another embodiment of the invention, R¹¹ and/or R¹² represents a C₁-C₆ alkyl group optionally substituted by at least one substituent (e.g. one, two, three or four substituents independently) selected from amino, hydroxyl, C₁-C₄ alkoxy, C₁-C₂ alkoxycarbonyl, C₁-C₂ alkylcarbonylamino and a 3- to 6-membered saturated or unsaturated ring optionally comprising one or two ring heteroatoms selected from nitrogen and oxygen and optionally further comprising a bridging group (in particular, cyclopropyl, bicyclo[2.2.1]heptyl, phenyl or tetrahydrofuranyl), the ring being optionally substituted with at least one substituent (e.g. one, two or three substituents independently) selected from oxo (e.g. to form a 2,5-dioxoimidazolidinyl ring) and C₁-C₂ alkyl.

In an embodiment of the invention, R¹¹ and R¹² together with the nitrogen atom to which they are attached form a 4- to 7-membered saturated heterocyclic ring that optionally further comprises a ring nitrogen, oxygen or sulphur atom (e.g. pyrrolidinyl, piperidinyl, piperazinyl, morpholinyl or thiomorpholinyl) and that is optionally fused to a benzene ring to form a 8- to 1 l-membered ring system (e.g. dihydroisoquinolinyl or dihydroisoindolyl), the heterocyclic ring or ring system being optionally substituted with at least one substituent (e.g. one, two,three or four substituents independently) selected from halogen (e.g. fluorine, chlorine, bromine or iodine), hydroxyl, amido, C₁-C₆, preferably C₁-C₄, alkyl (e.g. methyl, ethyl, n-propyl, isopropyl, n-butyl, isobutyl, tert-butyl, n-pentyl or n-hexyl), C₁-C₆, preferably C₁-C₄, hydroxyalkyl (e.g. —CH₂OH, —CH₂CH₂OH, —CH₂CH₂CH₂OH or —CH(OH)CH₃), C₁-C₆, preferably C₁-C₄, alkoxy (e.g. methoxy, is ethoxy, n-propoxy or n-butoxy), C₁-C₆, preferably C₁-C₄, alkoxycarbonyl (e.g. methoxycarbonyl or ethoxycarbonyl), C₁-C₆, preferably C₁-C₄, haloalkyl (e.g. trifluoromethyl), di-C₁-C₆, preferably C₁-C₄, alkylamino (e.g. dimethylamino), C₁-C₆, preferably C₁-C₄, alkylcarbonylamino (e.g. methylcarbonylamino or ethylcarbonylamino), di-C₁-C₆, preferably C₁-C₄, alkylaminocarbonyl (e.g. dimethylaminocarbonyl), phenyl, halophenyl (e.g. fluorophenyl or chlorophenyl), phenylcarbonyloxy and hydroxydiphenylmethyl.

In an embodiment of the invention, R¹¹ and R¹² together with the nitrogen atom to which they are attached form a 5- to 6-membered saturated heterocyclic ring that optionally further comprises a ring nitrogen, oxygen or sulphur atom and that is optionally fused to a benzene ring to form a 9- to 10-membered ring system, the heterocyclic ring or ring system being optionally substituted with one or two substituents independently selected from fluorine, hydroxyl, amido, C₁-C₂ alkyl, C₁-C₂ hydroxyalkyl, C₁-C₂ alkoxy, C₁-C₂ alkoxycarbonyl, C₁-C₂ haloalkyl, di-C₁-C₂ alkylamino, C₁-C₂ alkylcarbonylamino, di-C₁-C₂ alkylaminocarbonyl, phenyl, chlorophenyl, phenylcarbonyloxy and hydroxydiphenylmethyl.

In another embodiment of the invention, R¹¹ and R¹² together with the nitrogen atom to which they are attached form a heterocyclic ring or ring system selected from pyrrolidinyl, piperidinyl, piperazinyl, morpholinyl, thiomorpholinyl, dihydroisoquinolinyl and dihydroisoindolyl, the heterocyclic ring or ring system being optionally substituted with one or two substituents independently selected from fluorine, hydroxyl, amido, methyl, hydroxymethyl, 2-hydroxyethyl, methoxy, methoxycarbonyl, trifluoromethyl, dimethylamino, methylcarbonylamino, dimethylaminocarbonyl, phenyl, chlorophenyl, phenylcarbonyloxy and hydroxydiphenylmethyl.

R^{12a} represents a hydrogen atom or a C₁-C₆, preferably C₁-C₄, alkyl (e.g. methyl, ethyl, n-propyl, isopropyl, n-butyl, isobutyl, tert-butyl, n-pentyl or n-hexyl) group.

In an embodiment of the invention, R^{12a} represents a hydrogen atom or methyl group.

R¹³ represents a C₁-C₆, preferably C₁-C₄, alkyl group (e.g. methyl, ethyl, n-propyl, isopropyl, n-butyl, isobutyl, tert-butyl, n-pentyl or n-hexyl), amino or phenyl group.

In an embodiment of the invention:
m is 1;
R¹ represents halogen;
X represents a bond, —CH₂— or —O—, Y represents a bond, —CH₂— or —O— and Z represents —CH₂— or —O—, provided that X, Y and Z are different to one another;
n is 0;
q is 1;
R³ represents —NHC(O)R¹⁰ or —C(O)NR¹¹R¹²;
R⁴, R⁵, R⁶, R⁷ and R⁸ each represent hydrogen or methyl;
t is 0 or 1;
R⁹ represents halogen or hydroxyl;
R¹⁰ represents methyl; and
R¹¹ and R¹² each independently represent hydrogen or methyl.

Examples of compounds of the invention include:
2-({(2S)-3-[(5-Chloro-3H-spiro[1-benzofuran-2,1′-cyclohexan]4′-yl)amino]-2-hydroxypropyl}oxy)-4-hydroxy-N-methylbenzamide,
N-2-({(2S)-3-[5-Chloro-3H-spiro[1-benzofuran-2,1′-cyclohexan]-4′-yl)amino]-2-hydroxypropyl}oxy)-4-fluorophenyl]acetamide,
2-({(2S)-3-[(5-Chloro-3H-spiro[1-benzofuran-2,1′-cyclohexan]-4′-yl)amino]-2-hydroxypropyl}oxy)-N-methylbenzamide,
N-[2-({(2S)-3-[(5-Chloro-3H-spiro[1-benzofuran-2,1′-cyclohexan]-4′-yl)amino]-2-hydroxypropyl}oxy)-4-hydroxyphenyl]acetamide,
N-[2-({(2S)-3-[(5-Chloro-3H-spiro[1-benzofuran-2,1′-cyclohexan]4′-yl)amino]-2-hydroxy-2-methylpropyl}oxy)-4-hydroxyphenyl]acetamide (trifluoro acetate),
   and pharmaceutically acceptable salts and solvates of any one thereof.

The present invention further provides a process for the preparation of a compound of formula (I) or a pharmaceutically acceptable salt or solvate thereof as defined above which comprises,
(a) reacting a compound of formula 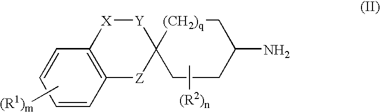
wherein m, R¹, n, R², q, X, Y and Z are as defined in formula (I), with a compound of formula 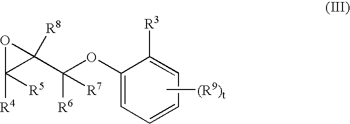
wherein R³, R⁴, R⁵, R⁶, R⁷, R⁸, t and R⁹ are as defined in formula (I); or
(b) reacting a compound of formula 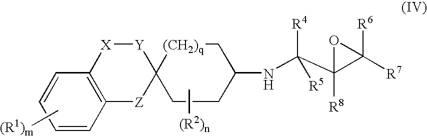
wherein m, R¹, n, R², q, X, Y, Z, , R⁴, R⁵, R⁶, R⁷ and R⁸ are as defined in formula (I), with a compound of formula 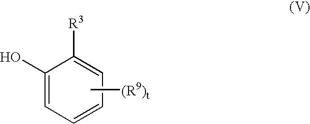
wherein R³, t and R⁹ are as defined in formula (I), in the presence of a suitable base; or
(c) when R³ represents —NHC(O)R¹⁰, reacting a compound of formula 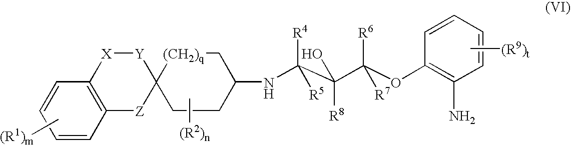
wherein m, R¹, n, R², q, X, Y, Z, R⁴, R⁵, R⁶, R⁷, R⁸, t and R⁹ are as defined in formula (I), with a compound of formula 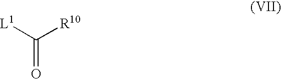
wherein L¹ represents a leaving group (e.g. a hydroxyl group or a halogen atom such as chlorine) and R¹⁰ is as defined in formula (I); or
(d) when R³ represents —C(O)NR¹¹R¹², reacting a compound of formula 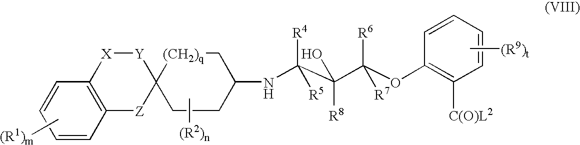
wherein L² represents a leaving group (e.g. a hydroxyl group or a halogen atom such as chlorine) and m, R¹, n, R², q, X, Y, Z, R⁴, R⁵, R⁶, R⁷, R⁸, t and R⁹ are as defined in formula (I), with a compound of formula (IX), NHR¹¹R¹², wherein R¹¹ and R¹² are as defined in formula (I); or
(e) when R³ represents —NHC(O)R¹⁰, R¹⁰ represents —NR¹⁴R¹⁵ and R¹⁴ and R¹⁵ both represent hydrogen, reacting a compound of formula (VI) as defined in (c) above with potassium cyanate;
and optionally after (a), (b), (c), (d) or (e) forming a pharmaceutically acceptable salt or solvate.

The processes of the invention may conveniently be carried out in a solvent, e.g. an organic solvent such as an alcohol (e.g. methanol or ethanol), a hydrocarbon (e.g. toluene) or tetrahydrofuran, dimethylformamide, N-methylpyrrolidinone or acetonitrile at a temperature of, for example, 0° C. or above such as a temperature in the range from 0, 5, 10, 15 or 20° C. to 100, 110 or 120° C.

Compounds of formulae (II), (III), (IV), (V), (VI), (VII), (VI) and (IX) are either commercially available, are known in the literature or may be prepared using known techniques.

It will be appreciated by those skilled in the art that in the processes of the present invention certain functional groups such as hydroxyl or amino groups in the reagents may need to be protected by protecting groups. Thus, the preparation of the compounds of formula (I) may involve, at an appropriate stage, the removal of one or more protecting groups.

The protection and deprotection of functional groups is described in ‘Protective Groups in Organic Chemistry’, edited by J. W. F. McOmie, Plenum Press (1973) and ‘Protective Groups in Organic Synthesis’, 3rd edition, T. W. Greene and P. G. M. Wuts, Wiley-Interscience (1999).

The compounds of formula (I) above may be converted to a pharmaceutically acceptable salt or solvate thereof, preferably an acid addition salt such as a hydrochloride, hydrobromide, phosphate, acetate, fumarate, maleate, tartrate, citrate, oxalate, methanesulphonate or p-toluenesulphonate.

Compounds of formula (I) are capable of existing in stereoisomeric forms. It will be understood that the invention encompasses the use of all geometric and optical isomers (including atropisomers) of the compounds of formula (I) and mixtures thereof including racemates. The use of tautomers and mixtures thereof also form an aspect of the present invention. Enantiomerically pure forms are particularly desired.

The compounds of formula (I) have activity as pharmaceuticals, in particular as modulators of chemokine receptor (especially MIP-l a chemokine receptor) activity, and may be used in the treatment of autoimmune, inflammatory, proliferative and hyperproliferative diseases and immunologically-mediated diseases including rejection of transplanted organs or tissues and Acquired Immunodeficiency Syndrome (AIDS).

Examples of these conditions are:
(1) (the respiratory tract) airways diseases including chronic obstructive pulmonary disease (COPD) such as irreversible COPD; asthma, such as bronchial, allergic, intrinsic, extrinsic and dust asthma, particularly chronic or inveterate asthma (e.g. late asthma and airways hyper-responsiveness); bronchitis; acute, allergic, atrophic rhinitis and chronic rhinitis including rhinitis caseosa, hypertrophic rhinitis, rhinitis purulenta, rhinitis sicca and rhinitis medicamentosa; membranous rhinitis including croupous, fibrinous and pseudomembranous rhinitis and scrofoulous rhinitis; seasonal rhinitis including rhinitis nervosa (hay fever) and vasomotor rhinitis; sarcoidosis, farmer's lung and related diseases, fibroid lung and idiopathic interstitial pneumonia;
(2) (bone and joints) rheumatoid arthritis, seronegative spondyloarthropathies (including ankylosing spondylitis, psoriatic arthritis and Reiter's disease), Behcet's disease, Sjogren's syndrome and systemic sclerosis;
(3) (skin) psoriasis, atopical dermatitis, contact dermatitis and other eczmatous dermitides, seborrhoetic dermatitis, Lichen planus, Pemphigus, bullous Pemphigus, Epidermolysis bullosa, urticaria, angiodermas, vasculitides, erythemas, cutaneous eosinophilias, uveitis, Alopecia areata and vernal conjunctivitis;
(4) (gastrointestinal tract) Coeliac disease, proctitis, eosinopilic gastro-enteritis, mastocytosis, Crohn's disease, ulcerative colitis, food-related allergies which have effects remote from the gut, e.g., migraine, rhinitis and eczema;
(5) (other tissues and systemic disease) multiple sclerosis, atherosclerosis, Acquired Immunodeficiency Syndrome (AIDS), lupus erythematosus, systemic lupus, erythematosus, Hashimoto's thyroiditis, myasthenia gravis, type 1 diabetes, nephrotic syndrome, eosinophilia fascitis, hyper IgE syndrome, lepromatous leprosy, sezary syndrome and idiopathic thrombocytopenia pupura;
(6) (allograft rejection) acute and chronic following, for example, transplantation of kidney, heart, liver, lung, bone marrow, skin and comea; and chronic graft versus host disease;
(7) cancers, especially non-small cell lung cancer (NSCLC) and squamous sarcoma;
(8) diseases in which angiogenesis is associated with raised chemokine levels; and
(9) cystic fibrosis, stroke, re-perfusion injury in the heart, brain, peripheral limbs and sepsis.

Thus, the present invention provides a compound of formula (I), or a pharmaceutically-acceptable salt or solvate thereof, as hereinbefore defined for use in therapy.

In a further aspect, the present invention provides the use of a compound of formula (I), or a pharmaceutically acceptable salt or solvate thereof, as hereinbefore defined in the manufacture of a medicament for use in therapy.

In the context of the present specification, the term “therapy” also includes “prophylaxis” unless there are specific indications to the contrary. The terms “therapeutic” and “therapeutically” should be construed accordingly.

The invention also provides a method of treating an inflammatory disease which comprises administering to a patient in need thereof a therapeutically effective amount of a compound of formula (I), or a pharmaceutically acceptable salt or solvate thereof, as hereinbefore defined.

The invention still further provides a method of treating an airways disease which comprises administering to a patient in need thereof a therapeutically effective amount of a compound of formula (I), or a pharmaceutically acceptable salt or solvate thereof, as hereinbefore defined.

For the above-mentioned therapeutic uses the dosage administered will, of course, vary with the compound employed, the mode of administration, the treatment desired and the disorder indicated. The daily dosage of the compound of formula (I) may be in the range from 0.001 mg/kg to 30 mg/kg.

The compounds of formula (I) and pharmaceutically acceptable salts and solvates thereof may be used on their own but will generally be administered in the form of a pharmaceutical composition in which the formula (I) compound/salt/solvate (active ingredient) is in association with a pharmaceutically acceptable adjuvant, diluent or carrier. Depending on the mode of administration, the pharmaceutical composition will preferably comprise from 0.05 to 99% w (per cent by weight), more preferably from 0.05 to 80% w, still more preferably from 0.10 to 70% w, and even more preferably from 0.10 to 50% w, of active ingredient, all percentages by weight being based on total composition.

The present invention also provides a pharmaceutical composition comprising a compound of formula (I), or a pharmaceutically acceptable salt or solvate thereof, as hereinbefore defined, in association with a pharmaceutically acceptable adjuvant, diluent or carrier.

The invention further provides a process for the preparation of a pharmaceutical composition of the invention which comprises mixing a compound of formula (I), or a pharmaceutically acceptable salt or solvate thereof, as hereinbefore defined, with a pharmaceutically acceptable adjuvant, diluent or carrier.

The pharmaceutical compositions may be administered topically (e.g. to the skin or to the lung and/or airways) in the form, e.g., of creams, solutions, suspensions, heptafluoroalkane aerosols and dry powder formulations; or systemically, e.g. by oral administration in the form of tablets, capsules, syrups, powders or granules; or by parenteral administration in the form of solutions or suspensions; or by subcutaneous administration; or by rectal administration in the form of suppositories; or transdermally.

The invention will now be further explained by reference to the following illustrative examples, in which ¹H NMR spectra were recorded on Varian Unity Inova 400. The central solvent peak of chloroform-d (δ_{H} 7.27 ppm), acetone-d₆ (δ_{H} 2.05 ppm), DMSO-d₆ (δ_{H} 2.50 ppm), or methanol-d₄ (δ_{H} 4.87 ppm) were used as internal standard. Low resolution mass spectra and accurate mass determination were recorded on a Hewlett-Packard 1100 LC-MS system equipped with APCI/ESI ionisation chambers.

All solvents and commercial reagents were laboratory grade and used as received.

The nomenclature used for the compounds was generated with ACD/IUPAC Name Pro.

The abbreviations or terms used in the examples have the following meanings:
THF: tetrahydrofuran
NH₄Cl: ammonium chloride
Na₂SO₄: sodium sulphate
NaH: sodium hydride
DMF: N,N-dimethylformamide
H₂O: water
CF₃CO₂H: trifluoroacetic acid
K₂CO₃: potassium carbonate
CH₂Cl₂: dichloromethane
NH₄OH: ammonium hydroxide
CH₃CN: acetonitrile
psi: pounds per square inch
Cs₂CO₃: caesium carbonate
HCl: hydrochloric acid
NaHCO₃: sodium hydrogencarbonate
Et₃N: triethylamine
DMAP: 4-dimethylarninopyridine
NaOEt: sodium ethoxide

### EXAMPLES

### Intermediate compound: (5-Chloro-3H-spiro[1-benzofuran-2,1′-cyclohexan]-4′-yl)amine

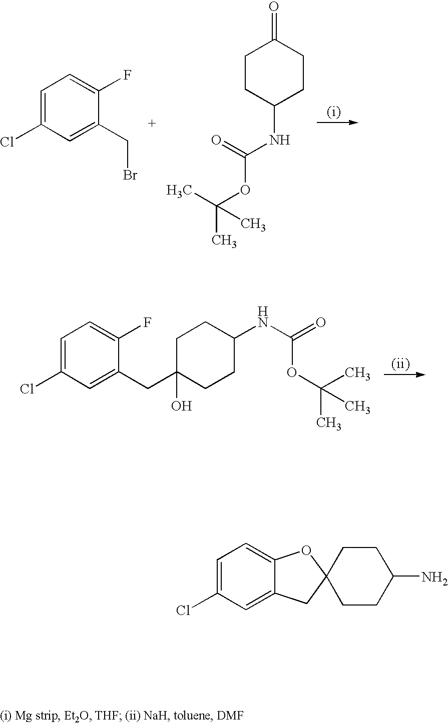 Step I
tert-Butyl[4-(5-chloro-2-fluorobenzyl)-4-hydroxycyclohexyl]carbamate

To a suspension of magnesium strip (283.6 mg, 11.67 mmol) in diethyl ether (4 mL) was added a piece of iodine followed by 0.3 mL of 2-(bromomethyl)-4-chloro-1-fluorobenzene under nitrogen atmosphere. A high intensity heat gun was applied to initiate the reaction, then 2-(bromomethyl)-4-chloro-1-fluorobenzene (2.61 g, 11.67 mmol) in diethyl ether (4.5 mL) was added slowly at such a speed that a gentle reflux was maintained. After the addition was completed, the reaction mixture was refluxed for 3 hours, cooled to room temperature and a solution of tert-butyl (4-oxocyclohexyl)carbamate (2.49 g, 11.67 mmol) in diethyl ether (9 mL) and THF (9 mL) was added slowly with vigorous stirring. After the addition was completed, the reaction mixture was left at room temperature for 3 hours. Aqueous NH₄Cl (20 mL) was added and the mixture was stirred at room temperature overnight, extracted with ethyl acetate, washed with H₂O, dried over Na₂SO₄, filtered and concentrated in vacuo. The residue was purified by silica gel flash chromatography (0-30% ethyl acetate in petroleum benzene) to give the subtitled compound (1.4 g).

Step H

(5-Chloro-3H-spiro[1-benzofuran-2,1′-cyclohexan]4′-yl)amine

A mixture of tert-butyl[4-(5-chloro-2-fluorobenzyl)-4-hydroxycyclohexyl]carbamate (1.4 g, 3.91 mmol) and NaH (55%, 511 mg, 11.73 mmol) in toluene (21 mL) was heated at 110° C. for 5 minutes. DMF (7 mL) was added and the mixture was stirred at 110 IC for 30 minutes before allowing to cool to room temperature. The reaction mixture was partitioned between ethyl acetate and H₂O. The organic layer was dried over Na₂SO₄, filtered and concentrated. The residue was purified by HPLC (10-45% acetonitrile in H₂O, 0.1% CF₃CO₂H) to give the corresponding trifluoroacetate salt which was converted to the free base to give the titled compound (60 mg).

¹H-NMR (CD₃OD, 400 MHz): δ 7.15 (s, 1H), 7.06 (dd, J=2.1, 8.5 Hz, 1H); 6.65 (d, J=8.5, Hz, 1H); 3.27 (m, 1H); 3.11 (s, 2H); 2.15-2.05 (m, 2H); 2.00-1.91 (m, 2H); 1.90-1.80 (m, 2H); 1.75-1.56 (m, 2H).

APCI-MS: m/z 238(MH⁺).

### Example 1

### 2-({(2S)-3-[(5-Chloro-3H-spiro[1-benzofuran-2,1′-cyclohexan]-4′-yl)amino]-2-hydroxypropyl}oxy)-4-hydroxy-N-methylbenzamide

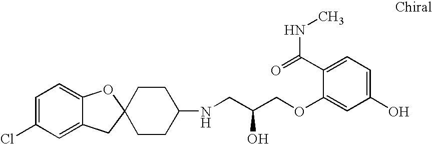 Step I
Methyl 2-hydroxy-4[(4-methoxybenzyl)oxy]benzoate

A mixture of methyl 2,4-dihydroxybenzoate (3.36 g, 20.0 mmol), p-methoxybenzyl chloride (3.29 g, 21.0 mmol) and K₂CO₃ (2.9 g, 21.0 mmol) in acetone (40 mL) was refluxed over night, cooled to room temperature, filtered and concentrated in vacuo. The residue was dissolved in ethyl acetate and washed with H₂O. The organic layer was dried over Na₂SO₄, filtered and concentrated. The residue was crystallized from methanol to give the sub titled compound (2.5 g).

¹H-NMR (CDCl₃, 400 MHz): δ 5.76 (d, J=8.9 Hz, 1H); 7.39 (m, 2H); 6.94 (m, 2H); 6.55 (d, J=2.5 Hz, 1H); 6.52 (dd, J=2.5, 8.9 Hz, 1H); 5.00 (s, 2H); 3.99 (s, 3H); 3.84 (s, 3H).

Reference: V. Percec, D. Tomazos J. Mater. Chem. 1993, 3, 643-650.

Step II

2-Hydroxy-4-[(4-methoxybenzyl)oxy]-N-methylbenzamide

To a suspension of methyl 2-hydroxy-4[(4-methoxybenzyl)oxy]benzoate (500 mg, 1.73 mmol) in methanol (15 mL) was added 40% aqueous methyl amine (3 mL) at 0° C. and the reaction mixture was stirred at room temperature over the weekend. The volatiles were removed in vacuo to give the subtitled compound (500 mg).

¹H-NMR (DMSO-d₆, 400 MHz): δ 8.60 (m, 1H); 7.70 (d, J=8.8 Hz, 1H); 7.38-7.33 (m, 2H); 6.96-6.91 (m, 2H); 6.49 (dd, J=2.6, 8.8 Hz, 1H); 6.42 (d, J=2.6 Hz, 1H); 5.00 (s, 2H); 3.75 (s, 3H); 2.77 (d, J=4.6 Hz, 3H).

APCI-MS: m/z 288(MH⁺).

Step III

4-[(4-Methoxybenzyl)oxy]-N-methyl-2-[(2S)-oxiran-2-ylmethoxy]benzaniide

A mixture of (2S)-oxiran-2-ylmethyl 3-nitrobenzenesulfonate (151 mg, 0.584 mmol), 2-hydroxy-4-[(4-methoxybenzyl)oxy]-N-methylbenzamide (168 mg, 0.584 mmol) and Cs₂CO₃ (228 mg, 0.7 mmol) in DMF (4 mL) was stirred at room temperature over night. The reaction mixture was partioned between ethyl acetate and H₂O. The organic layer was dried over Na₂SO₄, filtered and concentrated. The residue was purified by silica gel flash chromatography (0-90% ethyl acetate in petroleum benzene) to give the subtitled compound (150 mg).

¹H-NMR (DMSO-d₆, 400 MHz): δ 5.90 (m, 1H); 7.75 (d, J=8.7 Hz, 1H); 7.35 (d, J=8.6 Hz, 2H); 6.96-6.91 (m, 2H); 6.74 (d, J=2.3 Hz, 1H); 6.69 (dd, J=2.3, 8.7 Hz, 1H); 5.12 (s, 2H); 4.48 (dd, J=2.5, 11.5 Hz, 1H); 4.02 (dd, J=6.0, 11.5 Hz, 1H); 3.75 (s, 3H); 3.42 (m, 1H); 2.86 (t, J=4.9 Hz, 1H); 2.79 (d, J=4.7 Hz, 3H); 2.73 (dd, J=2.7, 5.0 Hz, 1H).

APCI-MS: m/z 344(MH⁺).

Step IV

2-({(2S)-3-[(5-Chloro-3H-spiro[1-benzofuran-2,1′-cyclohexan]-4′-yl)amino]-2-hydroxypropyl}oxy)-4-[(4-methoxybenzyl)oxy]-N-methylbenzamide

A mixture of (5-chloro-3H-spiro[1-benzofuran-2,1′-cyclohexan]-4′-yl)amine (25 mg, 0.105 mmol) and 4-[(4-methoxybenzyl)oxy]-N-methyl-2-[(2S)-oxiran-2-ylmethoxy]benzamide (36.3 mg, 0.105 mmol) in ethanol (2 mL) was stirred at 80° C. overnight. The volatiles were removed in vacuo and the residue was purified by silica gel flash chromatography (0-2% methanol in CH₂Cl₂, 0.2% NH₄₀H) to give the subtitled compound (15 mg).

APCI-MS: m/z 581(MH⁺).

Step V

2-({(2S)-3-[(5-Chloro-3H-spiro[1-benzofuran-2,1′-cyclohexan]-4′-yl)amino]-2-hydroxypropyl}oxy)-4-hydroxy-N-methylbenzamide

2-({(2S)-3-[(5-chloro-3H-spiro[1-benzofuran-2,1′-cyclohexan]-4′-yl)amino]-2-hydroxypropyl}oxy)-4-[(4-methoxybenzyl)oxy]-N-methylbenzamide (15 mg, 0.026 mmol) was treated with 10% CF₃CO₂H in CH₂Cl₂ (3 mL) at room temperature for 20 minutes. The volatiles were removed in vacuo and the residue was purified by HPLC (10-50% CH₃CN in H₂O, 0.2% NH₄OH) to give the titled compound (6 mg).

¹H-NMR (CD₃OD, 400 MHz): δ 5.79 (d, J=8.6 Hz, 1H); 7.13 (m, 1 H); 7.02 (dd, J=2.3, 8.5 Hz, 1H); 6.61 (d, J=8.5 Hz, 1H); 6.51 (d, J=2.2 Hz, 1H); 6.47 (dd, J=2.2, 8.6 Hz, 1H); 4.214.04 (m, 3H); 3.07 (s, 2H); 2.92 (s, 3H); 2.87 (dd, J=4.3, 12.2 Hz, 1H); 2.77 (dd, J=7.5, 12.2 Hz, 1H); 2.69 (m, 1H); 2.01 (m, 2H); 1.90 (m, 2H); 1.78 (m, 2H); 1.39 (m, 2H).

APCI-MS: m/z 461 (MH⁺).

### Example 2

### N-2-({(2S)-3-[5-Chloro-3H-spiro[1-benzofuran-2,1′-cyclohexan]-4′-yl)amino]-2-hydroxypropyl}oxy)-4-fluorophenyl]acetamide

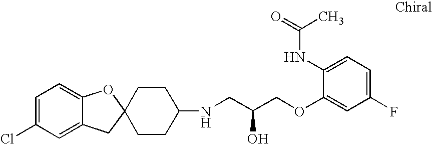 Step I
N-(4-Fluoro-2-hydroxyphenyl)acetamide

A mixture of 5-fluoro-2-nitrophenol (5 g, 31.8 mmol), acetic anhydride (4.86 g, 47.7 mmol) and platinum on carbon (5%, 200 mg) in methanol was hydrogenated at 35 psi for 3 hours. The catalyst was filtered off and the residue was purified by silica gel flash chromatography to give the subtitled compound (4.7 g).

¹H-NMR (CD₃OD, 300 MHz): δ 8.56-7.51 (m, 1H); 6.61-6.50 (m, 2H); 2.15 (s, 3H).

APCI-MS: m/z 170(MH⁺).

Step II

N-{4-Fluoro-2-[(2S)-oxiran-2-ylmethoxy]phenyl}acetamide

A mixture of N-(4-fluoro-2-hydroxyphenyl)acetamide (1.69 g, 10.0 mmol), (2S)-oxiran 2-ylmethyl-3-nitrobenzenesulfonate (2.59 g, 10.0 mmol) and Cs₂CO₃ (4.87 g, 15.0 mmol) in DMF (15 mL) was stirred at room temperature for 2 hours. The reaction mixture was partitioned between ethyl acetate and H₂O. The organic layer was dried over Na₂SO₄, filtered and concentrated. The residue was purified by silica gel flash chromatography to give the subtitled compound (1.35 g).

¹H-NMR (CDCl₃, 400 MHz): δ 5.33-8.29 (m, 1H); 7.71 (br. s, 1H); 6.74-6.66 (m, 2H); 4.39-4.36 (m, 1H); 3.95-3.90 (m, 1H); 3.41-3.39 (m, 1H); 2.99-2.97 (m, 1H); 2.80 (m, 1H).

APCI-MS: m/z 226(MH⁺).

Step III

N-2-({(2S)-3-[5-Chloro-3H-spiro[1-benzofuran-2,1′-cyclohexan]-4′-yl)amino]-2-hydroxypropyl}oxy)-4-fluorophenyl]acetamide

A mixture of (5-chloro-3H-spiro[1-benzofuran-2,1′-cyclohexan]-4′-yl)amine (11.6 mg, 0.049 mmol) and N-(4-fluoro-2-[(2S)-oxiran-2-ylmethoxy]phenyl}acetamide (11 mg, 0.049 mmol) in ethanol (1.5 mL) was stirred at 80° C. overnight. The volatiles were removed in vacuo and the residue was purified by silica gel flash chromatography (0-1.5% methanol in CH₂Cl₂, 0.2% NH₄₀H) to give the titled compound (10 mg).

¹H-NMR (CD₃OD, 400 MHz): δ 5.87 (dd, J=6.2, 8.9 Hz, 1H); 7.13 (m, 1H); 7.05 (dd, J=2.3, 8.5 Hz, 1H); 6.86 (dd, J=2.7, 10.5 Hz, 1H); 6.71-6.64 (m, 1H); 6.61 (d, J=8.5 Hz, 1H); 4.14-4.07 (m, 2H); 3.99 (dd, J=7.1, 10.6 Hz, 1H); 3.08 (s, 2H); 2.89 (dd, J=4.0, 12.0 Hz, 1H); 2.77 (dd, J=7.6, 12.0 Hz, 1H); 2.69 (m, 1H); 2.17 (s, 3H); 2.04 (m, 2H); 1.90 (m, 2H); 1.78 (m, 2H); 1.40 (m, 2H).

APCI-MS: m/z 463(MH⁺).

### Example 3

### 2-({(2S)-3-[(5-Chloro-3H-spiro[1-benzofuran-2,1′-cyclohexan]-4′-yl)amino]-2-hydroxypropyl}oxy)-N-methylbenzamide

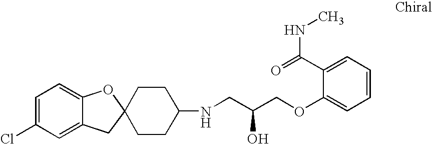 Step I
2-Hydroxy-N-methylbenzamide

A solution of methyl salicylate (5.16 mL, 40 mmol) in methanol (10 mL) was added dropwise to aqueous 40% methylamine (18.1 mL, 210 mmol) at 0° C. After the addition was completed the reaction mixture was stirred at room temperature overnight. The volatiles were removed in vacuo to give the subtitled compound (5.48 g).

¹H-NMR (CD₃OD, 400 MHz): δ 5.70 (dd, J=1.5, 7.9 Hz, 1H); 7.38-7.32 (m, 2H); 6.90-6.83 (m, 2H); 2.85 (s, 3H).

Step II

N-Methyl-2-[(2S)-oxiran-2-ylmethoxy]benzaniide

A mixture of (2S)-oxiran-2-ylmethyl-3-nitrobenzenesulfonate (388.5 mg, 1.50 mmol), 2-hydroxy-N-methylbenzamide (226.5 mg, 1.50 mmol) and Cs₂CO₃ (586 mg, 1.80 mmol) in DMF (6 mL) was stirred at room temperature overnight. The reaction mixture was partitioned between ethyl acetate and H₂O. The organic layer was dried over Na₂SO₄, filtered and concentrated. The residue was purified by silica gel flash chromatography (0-50% ethyl acetate in petroleum benzene) to give the subtitled compound (284 mg).

¹H-NMR (CDCl₃, 400 MHz): δ 5.39 (m, 1H); 7.90 (br.s, 1H); 7.06-6.98 (m, 2H); 6.95-6.89 (m, 1H); 4.38 (dd, J=2.5, 11.4 Hz, 1H); 3.98 (dd, J=6.0, 11.4 Hz, 1H); 3.40 (m, 1H); 2.97 (t, J=5.0 Hz, 1H); 2.81 (dd, J=2.7, 4.8 Hz, 1H); 2.21 (s, 3H).

APCI-MS: m/z 208(MH⁺).

Step III

2-({(2S)-3-[(5-Chloro-3H-spiro[1-benzofuran-2,1′-cyclohexan]-4′-yl)amino]-2-hydroxypropyl}oxy)-N-methylbenzamide

A mixture of (5-chloro-3H-spiro[1-benzofuran-2,1′-cyclohexan]4′-yl)amine (14 mg, 0.059 mmol) and N-methyl-2-[(2S)-oxiran-2-ylmethoxylbenzamide (12.2 mg, 0.059 mmol) in ethanol (1.5 mL) was stirred at 80° C. over night. The volatiles were removed in vacuo and the residue was purified by HPLC (10-50% CH₃CN in H₂O, 0.2% NH₄OH) to give the titled compound (5 mg).

¹H-NMR (CD₃OD, 400 MHz): δ 5.86 (dd, J=1.7, 7.7 Hz, 1H); 7.50-7.45 (m, 1H); 7.15 (m, 2H); 7.10-7.05 (m, 1H); 7.03 (dd, J=2.2,8.5Hz, 1H); 6.61 (d, J=8.5Hz, 1H); 4.24 (m, 1H); 4.15 (m, 2H); 3.07 (s, 2H); 2.95 (s, 3H); 2.89 (m, 1H); 2.80 (m, 1H); 2.69 (m, 1H); 2.02 (m, 2H); 1.90 (m, 2H); 1.75 (m, 2H); 1.39 (m, 2H).

APCI-MS: m/z 445(MH⁺).

### Example 4

### N-[2-({(2S)-3-[(5-Chloro-3H-spiro[1-benzofuran-2,1′-cyclohexan]-4′-yl)amino]-2-hydroxypropyl}oxy)-4-hydroxyphenyl]acetamide

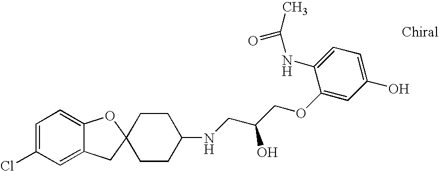 Step I
(1Z)-1-(2,4-Dihydroxyphenyl)ethanone oxime

1-(2,4-Dihydroxyphenyl)ethanone (4.5 g, 29.6 mmol) was dissolved in pyridine (17 mL). Hydroxylamine hydrochloride (2.1 g, 29.6 mmol) was added in small portions over 10 minutes. The reaction mixture was stirred at room temperature overnight. The reaction mixture was partitioned between ethyl acetate and H₂O. The organic layer was washed with H₂O, 0.2 M HCl and then concentrated. The oily residue was treated with water, evaporated to yield a white semi-solid residue which was treated with toluene and evaporated to give the subtitled compound (4.8 g) as a white solid.

APCI-MS: m/z 168(MH⁺).

Step II

2-Methyl-1,3-benzoxazol-6-ol

To a cooled (5° C.) solution of (1Z)-1-(2,4-dihydroxyphenyl)ethanone oxime (9.7 g, 57.7 mmol) in acetonitrile (65 mL) and dimethylacetamide (11 mL) was added phosphorous oxychloride (5.6 mL, 60.3 mmol) dropwise. The temperature was not allowed to exceed 10° C. during the addition. After 1 hour stirring at room temperature the yellow slurry was poured into a mixture of aqueous NaHCO₃ and ice. The resulting precipitate was filtered off and dried to give the subtitled compound (6.3 g).

¹H-NMR (DMSO-d₆, 400 MHz): δ 5.40 (d, 1H); 6.98 (s, 1H); 6.89 (d, 1H); 2.45 (s, 3H).

APCI-MS: m/z 150(MH⁺).

Step III

2-Methyl-1,3-benzoxazol-6-yl acetate

A slurry of methyl-1,3-benzoxazol-6-ol (7.1 g, 47.8 mmol) in THF 150 mL) was cooled to 10° C. and Et₃N (5.8 mL, 81.3 mmol) was added in one portion, followed by the addition of acetyl chloride (11.3 mL, 81.6 mmol) in small portions. After addition was completed the reaction mixture was stirred at room temperature overnight. The reaction mixture was partitioned between ethyl acetate and water. The organic layer was dried over Na₂SO₄, filtered and concentrated to give the subtitled compound (8.2 g).

¹H-NMR (DMSO-d₆, 400 MHz): δ 7.65 (d, 1H); 7.47 (s, 1H); 7.15 (d, 1H); 2.60 (s, 3H); 2.24 (s, 3H).

Step IV

4-(Acetylamino)-3-hydroxyphenyl acetate

To a solution of 2-methyl-1,3-benzoxazol-6-yl acetate (5.05 g, 28.8 mmol) in THF (100 mL) a mixture trifluoroacetic acid/water (4 ml/10 mL) was added. The reaction mixture was stirred at room temperature for 16 hours, then saurated aqueous NaHCO₃ (150 mL) was added. The mixture was extracted with ethyl acetate (150 mL), dried over Na₂SO₄, filtered and concentrated in vacuo to give the subtitled compound (4.0 g)

Step V

4-(Acetylamino)-3-[(2S)-oxiran-2-ylmethoxy]phenyl acetate

A solution of 4-(acetylamino)-3-hydroxyphenyl acetate (669 mg, 3.2 mmol), (2S)-oxiran-2-ylmethyl-3-nitrobenzenesulfonate (748 mg, 2.9 mmol) and Cs₂CO₃ (1.05 g, 3.2 mmol) in 1-methyl-pyrrolidinone (10 mL) was stirred at room temperature overnight. The reaction mixture was partitioned between ethyl acetate and water. The organic layer was dried over Na₂SO₄, filtered and concentrated to give a yellow oil which was suspended in methanol/diethyl ether. The precipitate was filtered off and dried to give the subtitled compound (296 mg).

¹H-NMR (CDCl₃, 400 MHz): δ 5.40 (d, 1H); 7.80 (s, 1H); 6.78 (m, 2H); 4.39 (m, 1H); 3.92 (m, 1H); 3.40 (m, 1H); 2.98 (t, 1H); 2.80 (m, 1H); 2.25 (s, 3H); 2.20 (s, 3H).

Step VI

N-[2-({(2S)-3-[(5-Chloro-3H-spiro[1-benzofuran-2,11-cyclohexan]-41-yl)amino]-2-hydroxypropyl}oxy)-4-hydroxyphenyl]acetamide

A mixture of (5-chloro-3H-spiro[1-benzofuran-2,1′-cyclohexan]-4′-yl)amine (14 mg, 0.06 mmol) and 4-(acetylamino)-3-[(2S)-oxiran-2-ylmethoxy]phenyl acetate (16 mg, 0.06 mmol) in ethanol (1.5 mL) was stirred at 80° C. over the weekend. The volatiles were removed in vacuo and the residue was purified by silica gel flash chromatography (0-3.5% methanol in CH₂Cl₂, 0.2% NHOH) to give the titled compound (15 mg).

¹H-NMR (CD₃OD, 400 MHz): δ 8.53 (d, J=8.6 Hz, 1H); 7.13 (m, 1H); 7.03 (dd, J=2.3, 8.5 Hz, 1H); 6.61 (d, J=8.5 Hz, 1H); 6.47 (d, J=2.5 Hz, 1H); 6.36 (dd, J=2.5, 8.6 Hz, 1H); 4-11-4.04 (m, 1H); 4.02 (dd, J=4.0, 9.8 Hz, 1H); 3.95 (dd, J=6.0, 9.8 Hz, 1H); 3.08 (s, 2H); 2.89 (dd, J=4.2, 12.2 Hz, 1H); 2.75 (dd, J=8.1, 12.2 Hz, 1H); 2.68 (m, 1H); 2.11 (s, 3H); 2.02 (m, 2H); 1.90 (m, 2H); 1.78 (m, 2H); 1.39 (m, 2H).

APCI-MS: m/z 461 (MH⁺).

### Example 5

### N-[2-({(2S)-3-[(5-Chloro-3H-spiro[1-benzofuran-2,1′-cyclohexan]-4′-yl)amino]-2-hydroxy-2-methylpropyl}oxy)-4-hydroxyphenyl]acetamide (trifluoro acetate)

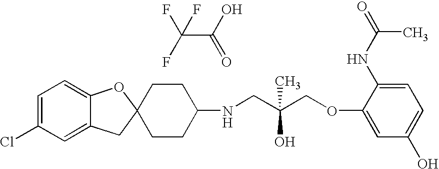 Step I
2-Methyl-1,3-benzoxazol-6-yl benzoate:

To a stirred suspension of 2-methyl-1,3-benzoxazol-6-ol (2.99 g, 20 mmol) in dichloromethane (50 mL) was added triethylamine (4.05 g, 5.58 mL, 40 mmol). A solution of benzoyl chloride (3.09 g, 2.56 mL, 22 mmol) in dichloromethane (20 mL) was added dropwise over about 10 minutes. The reaction mixture was stirred at room temperature for 2.5 hours, then washed with water (2×50 mL), and dried over Na₂SO₄, filtered and concentrated in vacuo to give the subtitled compound as colourless solid (5.05 g, 20 mmol, quant.).

¹H-NMR (400 MHz, CDCl₃): δ 8.22 (m, 2H), 7.66 (m, 2H), 7.53 (m, 2H), 7.40 (d, 1H), 7.16 (dd, 1H), 2.65 (s, 3H).

APCI-MS: m/z 254[MH⁺].

Step II

4-(Acetylamino)-3-hydroxyphenyl benzoate:

To a solution of 2-methyl-1,3-benzoxazol-6-yl benzoate (5.05 g, 20 mmol) in THF (100 mL) a mixture trifluoroacetic acid/water (4 ml/10 mL) was added. The reaction mixture was stirred at room temperature for 16 hours, then saurated aqueous NaHCO₃ (150 mL) was added. The mixture was extracted with ethyl acetate (150 mL), dried over Na₂SO₄, filtered and concentrated in vacuo to give the subtitled compound.

¹H-NMR (400 MHz, acetone-d₆): δ 9.76 (br.s, 1H), 9.32 (br.s, 1H), 8.15 (m, 2H), 7.71 (m, 1H), 7.60 (m, 2H), 7.47 (d, 1H), 6.85 (m, 1H), 6.75 (m, 1H), 2.20 (s, 3H).

APCI-MS: m/z 272[MH⁺].

Step III

[(2S)-2-Methyloxiranyl]methyl3-nitrobenzenesulfonate

To an oven-dried 1000 mL three-necked flask was added powdered activated molecular sieves (8.0 g, 4A) and CH₂Cl₂ (440 mL), D-(−)-diisopropyl tartrate (4 mL, 14.2 mmol) and 2-methyl-2-propene-1-ol (20 mL, 240 mmol) was added and the mixture was cooled to −20° C. Titanium tetraisopropoxide (3.5 mL, 11.9 mmol) was added with a few milliliters of CH₂Cl₂ and the mixture was stirred at −20° C. for 30 minutes. Cumene hydroperoxide (75 mL, 430 mmol) was added dropwise over 1.5 hours maintaining the temperature at −20° C. The mixture was stirred at this temperature overnight. Trimethyl phosphite (40 mL, 340 mmol) was added dropwise over 5 hours maintaining the temperature at −20° C. Triethylamine (50 m]L, 360 mmol) and DMAP (3.48 g, 28.5 mmol) was added followed by a solution of 3-nitrobenzenesulphonyl chloride (47 g, 212 mmol) in CH₂Cl₂ (400 mL). The temperature was raised to −10° C. and the mixture was stirred at this temperature overnight. After removing the external cooling vessel, the reaction mixture was filtered through celite. The organic phase was washed with 10% tartaric acid (500 mL), saturated NaHCO₃ (300 mL) and brine (300 mL). The organic layer was dried over MgSO₄ and concentrated in vacuo to give 150 g of a yellow oil. The crude material was purified by silica gel flash chromatography (0-50% ethyl acetate in heptane) to give the subtitled compound (48.8 g).

¹H-NMR (CDCl₃, 400 MHz): δ 8.79-8.75 (m, 1H); 8.52 (ddd, J=1.1, 2.3, 8.3 Hz, 1H); 8.25 (ddd, J=1.1, 1.8, 7.8 Hz, 1H); 7.81 (t, J=8.5 Hz, 1H); 4.28 (d, J=11.3 Hz, 1H); 4.05 (d, J=11.3 Hz, 1H); 2.73 (d, J=4.4 Hz, 1H); 2.67 (d, J=4.4 Hz, 1H); 1.56 (s, 3H).

Step IV:

4-(Acetylamino)-3-{[(2S)-2-methyloxiran-2-yl]methoxyl}phenyl benzoate

A mixture of 4-(acetylamino)-3-hydroxyphenyl benzoate (2.71 g, 10 mmol), [(2S)-2-methyloxiran-2-yl]methyl3-nitrobenzenesulfonate (2.73 g, 10 mmol) and Cs₂CO₃ (3.57 g, 11 mmol) in 1-methylpyrrolidin-2-one (35 mL) was stirred at room temperature overnight. The reaction mixture was partitioned between ethyl acetate and water. The organic layer was dried over Na₂SO₄, filtered and concentrated. The residue was purified by silica gel flash chromatography (ethyl acetate/n-heptane) to give the sub titled compound as a colourless solid (1.31 g, 3.9 mmol, 39%).

¹H-NMR (400 MHz, CDCl₃): δ 8.41 (d, 1H), 8.18 (m, 2H), 7.91 (br.s, 1H), 7.63 (m, 1H), 7.50 (m, 2H), 6.83 (m, 1H), 4.15 (d, J=10.8 Hz, 1H), 4.03 (d, J=10.8 Hz, 1H), 3.99 (d, J=10.8 Hz, 1H), 2.92 (d, J=4.6Hz, 1H), 2.78 (d, J=4.6 Hz, 1H), 2.22 (s, 3H), 1.48 (s, 3H).

APCI-MS: m/z 342[MH⁺].

Step V

N-[2-({(2S)-3-[(5-Chloro-3H-spiro[1-benzofuran-2,1′-cyclohexan]-4′-yl)amino]-2-hydroxy-2-methylpropyl}oxy-4-hydroxyphenyl]acetamide (trifluoro acetate)

A mixture of 5-chloro-3H-spiro[1-benzofuran-2,1′-cyclohexan]-4′-amine (20 mg, 0.084 mmol) and 4-(acetylamino)-3-{[(2S)-2-methyloxiran-2-yl]methoxy}phenyl benzoate (29 mg, 0.084 mmol) in ethanol (1.5 mL) was stirred at 80° C. over night, 2 drops conc NaOEt was added and the mixture was stirred at room temperature for 4 hours. The volatiles were removed in vacuo and the residue was purified by HPLC (10-80% acetonitrile in water, 0.1% CF₃CO₂H) to give the titled compound (20 mg).

¹H-NMR (400 MHz, CD₃OD): δ 8.19-7.13 (m, 2H); 7.08-7.03 (m, 1H); 6.67-6.62 (m, 1H); 6.50 (m, 1H); 6.43-6.39 (m,. 1H); 3.90 (m, 2H); 3.38-3.00 (m, 5H); 2.20 (m, 2H); 2.23 (s, 3H); 2.22-1.90 (m, 2H); 1.82 (m, 2H); 1.68 (m, 2H); 1.42 (s, 3H).

APCI-MS: m/z 475[MH⁺].

THP-1 Chemotaxis Assay

Introduction

The assay measures the chemotactic response elicited by MIP-1α chemokine in the human monocytic cell line THP-1. Compounds are evaluated by their ability to depress the chemotactic response to a standard concentration of MIP-1α chemokine.

Methods

Culture of THP-1 Cells

Cells are thawed rapidly at 37° C. from frozen aliquots and resuspended in a 25 cm flask containing 5 ml of RPMI-1640 medium supplemented with Glutamax and 10% heat inactivated fetal calf serum without antibiotics (RPMI+10% HIEFCS). At day 3 the medium is discarded and replaced with fresh medium.

THP-1 cells are routinely cultured in RPMI-1640 medium supplemented with 10% heat inactivated fetal calf serum and glutamax but without antibiotics. Optimal growth of the cells requires that they are passaged every 3 days and that the minimum subculture density is 4×10⁵ cells/ml.

Chemotaxis Assay

Cells are removed from the flask and washed by centrifugation in RPMI+10% HEFCS+glutamax. The cells are then resuspended at 2×10⁷ cells/ml in fresh medium (RPMI+10% HIFCS+glutamax) to which is added calcein-AM (5 μl of stock solution to 1 ml to give a final concentration of 5×10⁻⁶M). After gentle mixing the cells are incubated at 37° C. in a CO₂ incubator for 30 minutes. The cells are then diluted to 50 ml with medium and washed twice by centrifugation at 400×g. Labelled cells are then resuspended at a cell concentration of 1×10⁷ cells/ml and incubated with an equal volume of MIP-1α antagonist (10⁻¹⁰M to 10⁻⁶M final concentration) for 30 minutes at 37° C. in a humidified CO₂ incubator.

Chemotaxis is performed using Neuroprobe 96-well chemotaxis plates employing 8 μM filters (cat no. 101-8). Thirty microlitres of chemoattractant supplemented with various concentrations of antagonists or vehicle are added to the lower wells of the plate in triplicate. The filter is then carefully positioned on top and then 25 μl of cells preincubated with the corresponding concentration of antagonist or vehicle is added to the surface of the filter. The plate is then incubated for 2 hours at 37° C. in a humidified CO₂ incubator. The cells remaining on the surface are then removed by adsorption and the whole plate is centrifuged at 2000 rpm for 10 minutes. The filter is then removed and the cells that have migrated to the lower wells are quantified by the fluorescence of cell associated calcein-AM. Cell migration is then expressed in fluorescence units after subtraction of the reagent blank and values are standardized to % migration by comparing the fluorescence values with that of a known number of labelled cells. The effect of antagonists is calculated as % inhibition when the number of migrated cells is compared with vehicle.

1. A compound of formula 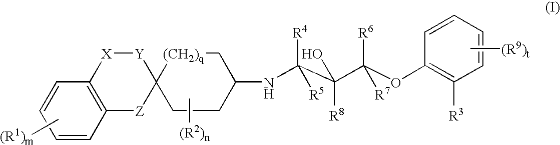
wherein
m is 0, 1, 2, 3 or 4;
each R¹ independently represents halogen, cyano, hydroxyl, C₁-C₆ alkyl, C₁-C₆ haloalkyl, C₁-C₆ alkoxy or sulphonamido;
either X represents a bond, —CH₂—, —O— or —C(O)— and Y represents a bond, —CH₂—, —O— or —C(O)—, or X and Y together represent a group —CH═C(CH₃)— or —C(CH₃)═CH—, and Z represents a bond, —O—, —NH— or —CH₂—, provided that only one of X, Y and Z can represent a bond at any one time and provided that X and Y do not both simultaneously represent —— or —C(O)—;
n is 0, 1 or 2;
each R² independently represents halogen or C₁-C₆ alkyl;
q is 0 or 1;
R³ represents —NHC(O)R¹⁰, —C(O)NR¹¹R¹² or —COOR^{12a};
R⁴, R⁵, R⁶, R⁷ and R⁸ each independently represent a hydrogen atom or a C₁-C₆ alkyl group;
t is 0, 1 or 2;
each R⁹ independently represents halogen, cyano, hydroxyl, carboxyl, C₁-C₆ alkoxy, C₁-C₆ alkoxycarbonyl, C₁-C₆ haloalkyl, or C₁-C₆ alkyl optionally substituted by at least one substituent selected from carboxyl and C₁-C₆ alkoxycarbonyl;
R¹⁰ represents a group C₁-C₆ alkyl, C₂-C₆ alkenyl, C₃-C₆ cycloalkyl, adamantyl, C₅-C₆ cycloalkenyl, phenyl or a saturated or unsaturated 5- to 10-membered heterocyclic ring system comprising at least one ring heteroatom selected from nitrogen, oxygen and sulphur, each of which may be optionally substituted by one or more substituents independently selected from nitro, hydroxyl, oxo, halogen, carboxyl, C₁-C₆ alkyl, C₁-C₆ alkoxy, C₁-C₆ alkylthio, C₁-C₆ alkylcarbonyl, Cₗ-C₆ alkoxycarbonyl, phenyl and —NHC(O)—R¹³, or
R¹⁰ represents a group —NR¹⁴R¹⁵ or —O—R¹⁶;
R¹¹ and R¹² each independently represent (i) a hydrogen atom, (ii) a 3- to 6-membered saturated or unsaturated ring optionally comprising at least one ring heteroatom selected from nitrogen, oxygen and sulphur and optionally further comprising a bridging group, the ring being optionally substituted with at least one substituent selected from halogen, hydroxyl, C₁-C₆ alkyl, C₁-C₆ hydroxyalkyl and C₁-C₆ haloalkyl, (iii) a C₁-C₆ alkyl group optionally substituted by at least one substituent selected from halogen, amino (—NH₂), hydroxyl, C₁-C₆ haloalkyl, carboxyl, C₁-C₆ alkoxy, C₁-C₆ alkoxycarbonyl, C₁-C₆ alkylcarbonylamino and a 3- to 6-membered saturated or unsaturated ring optionally comprising at least one ring heteroatom selected from nitrogen, oxygen and sulphur and optionally further comprising a bridging group, the ring being optionally substituted with at least one substituent selected from halogen, hydroxyl, oxo C₁-C₆ alkyl, C₁-C₆ hydroxyalkyl and C₁-C₆ haloalkyl, or (iv) C₁-C₆ alkylsulphonyl, or
R¹¹ and R¹² together with the nitrogen atom to which they are attached form a 4- to 7-membered saturated heterocyclic ring that optionally further comprises a ring nitrogen, oxygen or sulphur atom and that is optionally fused to a benzene ring to form a 8- to 11-membered ring system, the heterocyclic ring or ring system being optionally substituted with at least one substituent selected from halogen, hydroxyl, amido (—CONH₂), C₁-C₆ alkyl, C₁-C₆ hydroxyalkyl, C₁-C₆ alkoxy, C₁-C₆ alkoxycarbonyl, C₁-C₆ haloalkyl, C₁-C₆ alkylamino, di-C₁-C₆ alkylamino, C₁-C₆ alkylcarbonyl, C₁-C₆ alkylcarbonylamino, C₁-C₆ alkylaminocarbonyl, di-C₁-C₆ alkylaminocarbonyl, phenyl, halophenyl, phenylcarbonyl, phenylcarbonyloxy and hydroxydiphenylmethyl;
R^{12a} represents a hydrogen atom or a C₁-C₆ alkyl group;
R¹³ represents a C₁-C₆ alkyl, amino (—NH₂) or phenyl group;
R¹⁴ and R¹⁵ each independently represent a hydrogen atom, or a group C₁-C₆ alkyl, C₁-C₆ alkylsulphonyl, phenyl or a saturated or unsaturated 5- to 10-membered heterocyclic ring system comprising at least one ring heteroatom selected from nitrogen, oxygen and sulphur, each group being optionally substituted as defined above for R¹⁰, or
R¹⁴ and R¹⁵ together with the nitrogen atom to which they are attached form a 4- to 7-membered saturated heterocyclic ring that optionally further comprises a ring nitrogen, oxygen or sulphur atom, the heterocyclic ring being optionally substituted by at least one hydroxyl; and
R¹⁶ represents a hydrogen atom, or a group C₁-C₆ alkyl, phenyl or a saturated or unsaturated 5- to 10-membered heterocyclic ring system comprising at least one ring heteroatom selected from nitrogen, oxygen and sulphur, each group being optionally substituted as defined above for R¹⁰;
or a pharmaceutically acceptable salt or solvate thereof.

2. A compound according to claim 1, wherein X aid Y have the meanings shown in the following table:

| X | Y |
|---|---|
| bond | O |
| O | bond |
| CH₂ | bond |
| bond | CH₂ |

3. A compound according to claim 1, wherein Z represents —O— or —CH₂—.

4. A compound according to claim 1, wherein q is 1.

5. A compound according to claim 1, wherein R³ represents —NHC(O)R¹⁰ or —C(O)NR¹¹R¹².

6. A compound according to claim 1, wherein t is 1 and R⁹ is located in the para position with respect to R³.

7. A compound according to claim 1 selected from:
2-({(2S)-3-[(5-Chloro-3H-spiro[1-benzofuran-2,1′-cyclohexan]4′-yl)amino]-2-hydroxypropyl}oxy)-4-hydroxy-N-methylbenzamide,
N-2-({(2S)-3-[5-Chloro-3H-spiro[1-benzofuran-2,1′-cyclohexan]-4′-yl)amino]-2-hydroxypropyl}oxy)-4-fluorophenyl]acetamide,
2-({(2S)-3-[(5-Chloro-3H-spiro[1-benzofuran-2,1′-cyclohexan]-4′-yl)amino]-2-hydroxypropyl}oxy)-N-methylbenzamide,
N-[2-({(2S)-3-[(5-Chloro-3H-spiro[1-benzofuran-2,1′-cyclohexan]-4′-yl)amino]-2-hydroxypropyl}oxy)-4-hydroxyphenyl]acetamide,
N-[2-({(2S)-3-[(5-Chloro-3H-spiro[1-benzofuran-2,1′-cyclohexan]4′-yl)amino]-2-hydroxy-2-methylpropyl}oxy)-4-hydroxyphenyl]acetamide (trifluoro acetate),
and pharmaceutically acceptable salts and solvates of any one thereof.

8. A process for the preparation of a compound of formula (I) or a pharmaceutically acceptable salt or solvate thereof as defined in claim 1 which comprises,
(a) reacting a compound of formula 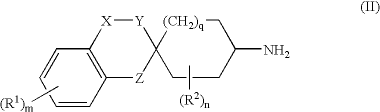
wherein m, R¹, n, R², q, X, Y and Z are as defined in formula (I), with a compound of formula 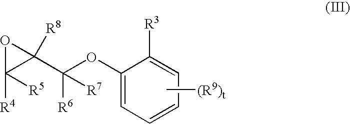
wherein R³, R⁴, R⁵, R⁶, R⁷, R⁸, t and R⁹ are as defined in formula (I); or
(b) reacting a compound of formula 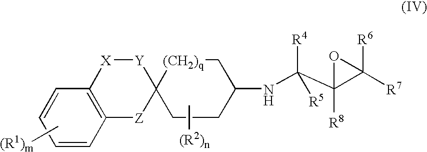
wherein m, R¹, n, R², q, X, Y, Z, , R⁴, R⁵, R⁶, R⁷ and R⁸ are as defined in formula (I), with a compound of formula 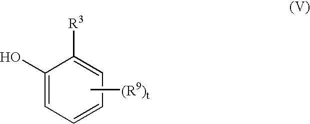
wherein R³, t and R⁹ are as defined in formula (I), in the presence of a suitable base; or
(c) when R³ represents —NHC(O)R¹⁰, reacting a compound of formula 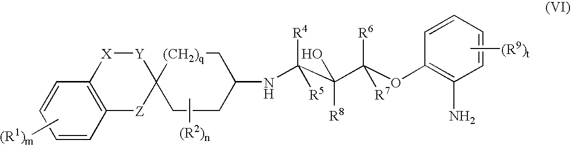
wherein m, R¹, n, R², q, X, Y, Z, R⁴, R⁵, R⁶, R⁷, R⁸, t and R⁹ are as defined in formula (I), with a compound of formula 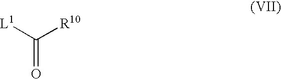
wherein L¹ represents a leaving group and R¹⁰ is as defined in formula (I); or
(d) when R³ represents —C(O)NR¹¹R¹², reacting a compound of formula 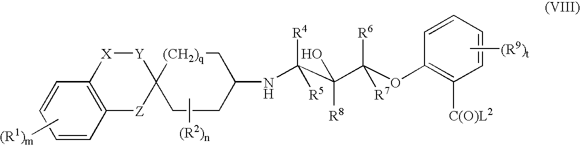
wherein L² represents a leaving group and m, R¹, n, R², q, X, Y, Z, R⁴, R⁵, R⁶, R⁷, R⁸, t and R⁹ are as defined in formula (I), with a compound of formula (IX), NHR¹¹R¹², wherein R¹¹ and R¹² are as defined in formula (I); or
(e) when R³ represents —NHC(O)R¹⁰, R¹⁰ represents —NR¹⁴R¹⁵ and R¹⁴ and R¹⁵ both represent hydrogen, reacting a compound of formula (VI) as defined in (c) above with potassium cyanate;
and optionally after (a), (b), (c), (d) or (e) forming a pharmaceutically acceptable salt or solvate.

9. A pharmaceutical composition comprising a compound of formula (I) or a pharmaceutically acceptable salt or solvate thereof as claimed in claim 1 in association with a pharmaceutically acceptable adjuvant, diluent or carrier.

10. A process for the preparation of a pharmaceutical composition as claimed in claim 9 which comprises mixing a compound of formula (I) or a pharmaceutically acceptable salt or solvate thereof as claimed in claim 1 with a pharmaceutically acceptable adjuvant, diluent or carrier.

11. (canceled)

12. A method of treating a disease or condition in which modulation of chemokine receptor activity is beneficial, the method comprising administering to a patient in need thereof a therapeutically effective amount of a compound of formula (I) or a pharmaceutically acceptable salt or solvate thereof as claimed in claim 1.

13. A method of treating rheumatoid arthritis, the method comprising administering to a patient in need thereof a therapeutically effective amount of a compound of formula (I) or a pharmaceutically acceptable salt or solvate thereof as claimed in claim 1.

14. A method of treating chronic obstructive pulmonary disease, the method comprising administering to a patient in need thereof a therapeutically effective amount of a compound of formula (I) or a pharmaceutically acceptable salt or solvate thereof as claimed in claim 1.

15. A method of treating asthma, the method comprising administering to a patient in need thereof a therapeutically effective amount of a compound of formula (I) or a pharmaceutically acceptable salt or solvate thereof as claimed in claim 1.

16. A method of treating multiple sclerosis, the method comprising administering to a patient in need thereof a therapeutically effective amount of a compound of formula (I) or a pharmaceutically acceptable salt or solvate thereof as claimed in claim 1.

17. A method of treating an inflammatory disease which comprises administering to a patient in need thereof a therapeutically effective amount of a compound of formula (I) or a pharmaceutically acceptable salt or solvate thereof as claimed in claim 1.

18. A method of treating an airways disease which comprises administering to a patient in need thereof a therapeutically effective amount of a compound of formula (I) or a pharmaceutically acceptable salt or solvate thereof as claimed in claim 1.
